(19) United States
(12) Patent Application Publication
(10) Pub. No.: US 2010/0185266 A1
(43) Pub. Date: Jul. 22, 2010

(54) Beauty Device

(76) Inventor: Shuhei Suzuki, Tokyo (JP)

Correspondence Address: WENDEROTH, LIND & PONACK, L.L.P., 1030 15th Street, N.W.,, Suite 400 East, Washington, DC, 20005-1503, US

(21) Appl. No.: 11/992,415

(22) PCT Filed: Apr. 23, 2007
(86) PCT No.: PCT/JP2007/058707

§ 371 Date: Mar. 21, 2008

(30) Foreign Application Priority Data
Dec. 8, 2004 (JP) 2006-331269

(51) Int. Cl. A61N 5/06

(52) U.S. Cl. 607/90

(57) ABSTRACT

A cosmetic appliance of simple structure, attaining easy handling, high safety and excellence in the effect of producing beautiful skin. The cosmetic appliance (1) is one including titanium roller (5) provided at its surface with a titanium dioxide layer and light irradiating part (7), characterized in that the light irradiating part (7) has two or more types of light emitting diodes (13) emitting lights with different wavelengths and is disposed along the lateral line of the titanium roller (5).

### TECHNICAL FIELD

The present invention relates to a beauty device, and more particularly to a beauty device that promotes skin activation, suppresses propagation of bacteria and exhibits excellent skin-beautifying effects.

### BACKGROUND ART

Patent document 1 discloses a beauty device that irradiates a human skin with light of light emitting diodes and performs ionization of a vitamin solution to be supplied to the skin.

Patent document 1: Unexamined Japanese Patent Document 2005-168894

### DISCLOSURE OF INVENTION

### Problems to be Solved by the Invention

However, in the beauty device disclosed in patent document 1, there are problems in which its configuration becomes complicated due to ionization of a vitamin solution and much time and effort are required to handle the device due to supplement of the vitamin solution.

Moreover, in patent document 1, suppression of propagation of bacteria on the surface of a human skin or sterilization thereof is insufficient, so that effects of preventing rough skin due to bacteria are low.

Therefore, beauty devices, which can be easily handled and safely used at home and by individuals, have been desired.

In view of the aforementioned circumstances, it is an object of the present invention is to provide a beauty device, which is easily configured, easily handled, and has high safety and excellent skin beautifying effects.

### Means for Solving the Problems

In order to solve the aforementioned problem, according to a first aspect of the present invention, a beauty device includes a titanium roller having a titanium dioxide layer on a surface, and a light irradiation section, wherein the light irradiation section has light emitting diodes of two or more types that emit light each having a different wavelength, and is placed along a side line of the titanium roller.

According to a second aspect of the present invention, the device further includes a holding section that is held with a hand, wherein the titanium roller and the light irradiation section are provided at a tip end side of the holding section, and the light irradiation section is placed at a rear end side rather than a rotating shaft of the titanium roller to irradiate in a tip end direction and irradiate the titanium roller with part of the irradiated light.

According to a third aspect of the present invention, in accordance with the second aspect of the present invention, the light emitting diodes include at least a light emitting diode that emits light having a wavelength ranging from red to yellow and a light emitting diode that emits near infrared light.

According to a fourth aspect of the present invention, in accordance with the first aspect of the present invention, each light emitting diode blinks at a frequency of two or more per second.

According to a fifth aspect of the present invention, the titanium roller is formed such that a surface of pure titanium is anodized.

### Effects of the Invention

According to the first aspect of the present invention, a human skin is stimulated by light irradiation of light emitting diodes (LEDs) to thereby promote activation of the skin and proliferation of bacteria adhered on the skin is prevented and sterilized by a photocatalysis function of titanium dioxide. It is possible to obtain a combined effect caused by the aforementioned light emitting diodes and the titanium dioxide, thereby making it possible to promote prevention of skin aging, suppression of pimples and skin whitening. Moreover, by the photocatalysis function of titanium dioxide aging material adhered on the skin can be decomposed, bioelectric current can be adjusted by ion emission, and moisture can be given to the skin by a hydrophilic function.

In the beauty device, the titanium roller is placed on the skin and simply moved along a skin surface to be rotated while emitting light from the light emitting diodes, thus resulting in a good operability.

Furthermore, no high voltage power is required because of the configuration with the titanium roller and the light emitting diodes, safe and easy configuration and easy handling are achieved

According to the second aspect of the present invention, the same effect as that in the first aspect of the present invention is provided and the titanium roller is irradiated with part of the light from the light emitting diodes and therefore it is possible to increase the photocatalysis function of titanium dioxide.

According to the third aspect of the present invention, the same effect as that in the second aspect of the present invention is provided, and light having a wavelength ranging from red to yellow is excellent in skin-beautifying effects since it has functions of preventing pimples, promoting activation of fibroblast cells, activating nerve blocks, and decomposing cholesterol. Moreover, near infrared light generally has high permeability to the skin to promote proliferation of the fibroblast cells, thus resulting in excellent skin-beautifying effects and making it possible to increase activation of titanium dioxide.

According to the fourth aspect of the present invention, the same effect as that in the first aspect of the present invention is provided, and the light emitting diodes are blinked to increase stimulation of the skin by light, thereby making it possible to exhibit excellent skin-beautifying effects such as prevention of wrinkles, blemishes and the like.

According to the fifth aspect of the present invention, the same effect as that in the fourth aspect of the present invention is provided, when a titanium dioxide layer is formed on the surface by anodizing pure titanium, the surface is formed in a thin film shape and is seen as a rainbow color due to light interference, resulting in a good appearance and an excellent design.

### BRIEF DESCRIPTION OF THE DRAWINGS

FIG. 1 is a cross-sectional view taken along line of A-A of a beauty device shown in FIG. 3;

FIG. 2 is a cross-sectional view taken along line of B-B of the beauty device shown in FIG. 3;

FIG. 3 is a perspective view showing an outline of a beauty device according to an embodiment of the present invention;

FIG. 4 is a perspective view showing a state in which the beauty device shown in FIG. 3 is used; and

FIG. 5 is a graph explaining wavelengths of light emitting diodes used in the embodiment of the present invention.

### DESCRIPTION OF THE SYMBOLS

1: Beauty device

3: Device main body

5: Titanium roller

7: Light irradiation section

13a, 13b: Light emitting diodes

15: Control section

17: Holding section

### BEST MODE FOR CARRYING OUT THE INVENTION

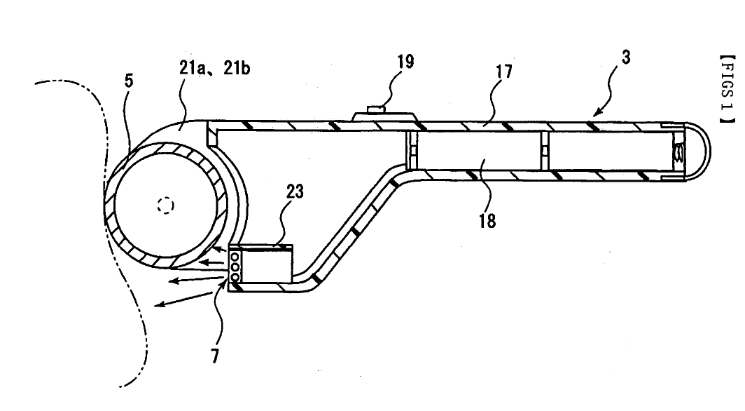

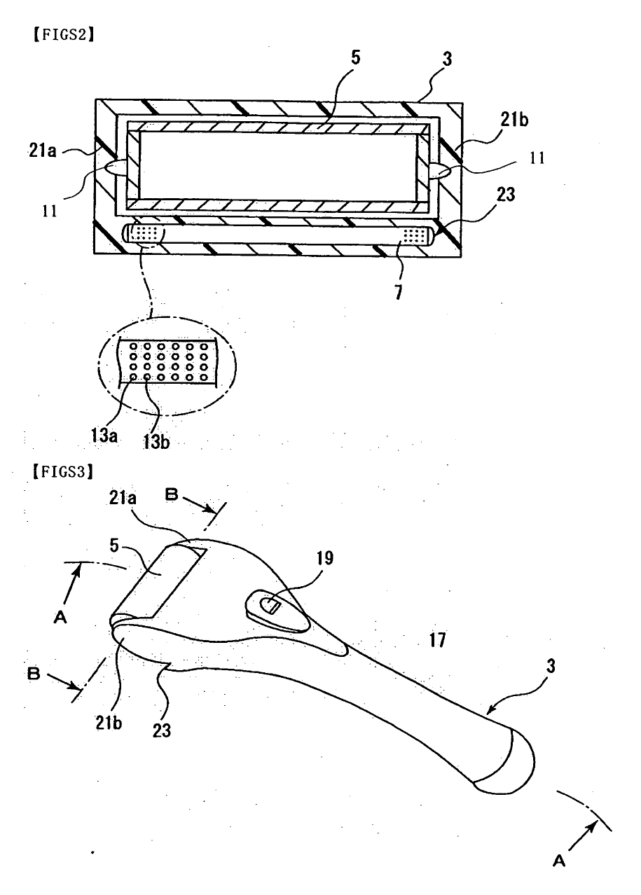

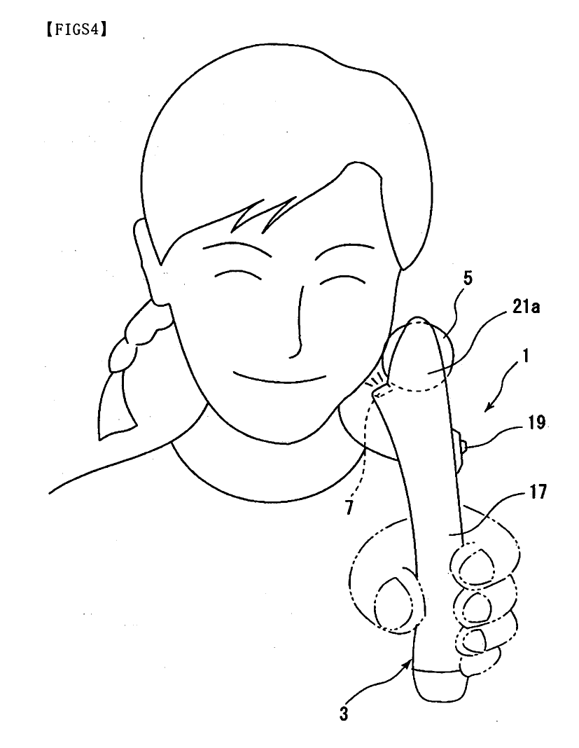

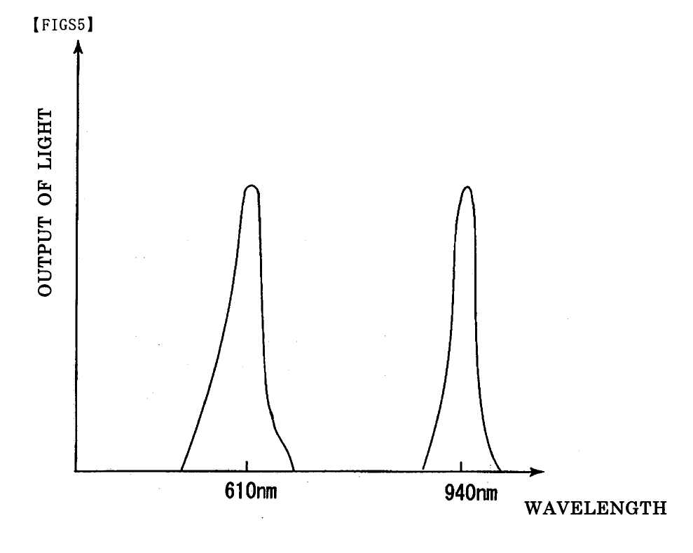

The following will explain an embodiment of the present invention with reference to the drawings. FIG. 1 is a cross-sectional view taken along line of A-A of a beauty device shown in FIG. 3, FIG. 2 is a cross-sectional view taken along line of B-B of the beauty device shown in FIG. 3, FIG. 3 is a perspective view showing an outline of a beauty device according to an embodiment of the present invention, FIG. 4 is a perspective view showing a state in which the beauty device shown in FIG. 3 is used, and FIG. 5 is a graph explaining wavelengths of light emitting diodes used in the embodiment of the present invention.

A beauty device 1 of this embodiment is individually used at home for the purpose of beauty skin care, and includes a device main body 3, a titanium roller 5, and a light irradiation section 7.

The titanium roller 5 is cylindrically shaped and has a titanium dioxide layer formed on a surface (side surface). The titanium dioxide layer is formed in a thin film shape by anodizing pure titanium. The titanium dioxide layer is formed in a thin film shape, so that its surface is seen as a rainbow color.

The cylindrical titanium roller 5 is freely rotatable in such a state that rotation shafts 11, 11 formed at both ends is pivotably supported by tip end portion of the device main body 3.

The light irradiation section 7 is provided close to the titanium roller 5 at the tip end side of the device main body 3. The light irradiation section 7 is formed as a unit, and can be freely attached and detached to and from an insertion section 23 to be described later.

The light irradiation section 7 has two types of light emitting diodes 13a and 13b each having a different light color to be emitted, and a large number of each of light emitting diodes 13a and 13b is provided. The light emitting diodes 13a of one type are those that emit light having a wavelength ranging from red to yellow and the diodes of the other type are those that emit near infrared light. More specifically, as shown in FIG. 5, the light emitting diodes 13a of one type emit light (orange near red) having a wavelength of around 610 nm, and the light emitting diodes 13b of the other type emit light (infrared region) having a wavelength of around 940 nm.

The light emitting diodes 13a, 13b of these types blink at a frequency of two or more per second and blinking interval is preferably 5 to 100 Hz, but in the present embodiment, control is made to achieve blinking at a frequency of 5 H by the control section 15.

There is no particular limitation in arrangement of the light emitting diodes 13a and 13b, but in the present embodiment, two types of light emitting diodes 13a and 13b are arranged to be adjacent to each other alternately.

Incidentally, a transparent light-scattering plate is provided on a light emission side of the light emitting diodes 13a and 13b to scatter light.

The device main body 3 is made of resin and includes a holding section 17, an ON/OFF switch 19 of the light irradiation section and titanium roller support sections 21 and 21b. Batteries 18 are inserted into the holding section 17. The insertion section 23 of the light irradiation section is provided between the titanium roller support sections 21 and 21b along a side surface of the titanium roller 5.

An explanation will be next given of a method for using the beauty device 1 and the function and effect according to the present embodiment. When using, a user holds the holding section of the device main body 3 with his/her hand and turns ON the ON/OFF switch 19 and puts the titanium roller 5 on the face skin as shown in FIG. 4. Then, when the device main body 3 is moved along the skin, the titanium roller rotatably moves on the skin face. Since the titanium roller has the thin film titanium dioxide layer on its surface (peripheral surface), the surface is seen as a rainbow color due to light interference, resulting in a good appearance.

Although the skin surface is irradiated with light emitted from the light emitting diodes 13a and 13b of the light irradiation section 7, the surface of the titanium roller 5 is irradiated with part of the light to activate the titanium dioxide formed on the surface of the titanium roller, thereby allowing photocatalysis of the titanium dioxide to be increased. Moreover, the light emitting diodes 13a and 13b blink at a predetermined frequency (frequency of 5 Hz in this embodiment), so that stimulation of the skin by light is increased, thereby making it possible to exhibit excellent skin-beautifying effects such as prevention of wrinkles, blemishes and the like.

According to the present embodiment, the skin is stimulated by light irradiation of the light emitting diodes 13a and 13b to thereby promote activation of the skin, and proliferation of bacteria adhered on the skin is prevented and sterilized by the photocatalysis function of the titanium dioxide.

It is possible to obtain a combined effect caused by the aforementioned light emitting diodes 13a and 13b and the titanium dioxide, thereby making it possible to promote prevention of skin aging, suppression of pimples and skin whitening. Moreover, aging material adhered on the skin can be decomposed by the photocatalysis function of titanium dioxide, bioelectric current can be adjusted by ion emission, and moisture can be given to the skin by a hydrophilic function.

The titanium roller 5 is placed on the skin and simply moved on the skin to be rotated, thus resulting in a good operability.

Furthermore, no high voltage power is required because of the configuration with the titanium roller and the light emitting diodes, safe and easy configuration and easy handling are achieved and easy usage can be achieved even at home.

Particularly, in the light irradiation section 7, light emitting diodes 13a of one type emit light having a wavelength ranging from red to yellow. In general, light having such a wavelength is excellent in skin-beautifying effects since it has functions of preventing pimples, promoting activation of fibroblast cells, activating nerve blocks, and decomposing cholesterol. Furthermore, the light emitting diodes 13b of the other type generally have high permeability to the skin to promote proliferation of the fibroblast cells, thus resulting in excellent skin-beautifying effects and making it possible to increase activation of titanium dioxide.

The present invention is not limited to the aforementioned embodiment, and can variously be modified without departing from the scope of the present invention.

For example, the light emitting diodes are not limited to 13a and 13b of two types including two wavelengths, and may be those of four types including yellow light, red light, near infrared and blue light. In the case of irradiation of four types of colors, the nervous blocks are further stimulated to allow blemishes, wrinkles and the like to be reduced effectively. In addition, light of multiple types is sufficiently and uniformly interfered with one another to allow irradiation, and therefore it is possible to obtain cosmetic effects such as improvement in pimple skin, moisture effects, improvement in ruddy face, pore tightening, etc. Furthermore, the light emitting diodes having wavelengths of 416 nm, 460 nm, 575 nm, 635 nm, 897 nm, and the like may be used.

Moreover, two light irradiation sections 7 may be provided, in which case one irradiates the peripheral surface of the titanium roller and the other irradiates the skin.

In the invention according to claims 1 to 3, the light emitting diodes 13a and 13b may not be blinked.

1-5. (canceled)

6. A beauty device comprising:
a holding section that is held with a hand;
a titanium roller having a titanium dioxide layer on a surface; and
a light irradiation section,
wherein the light irradiation section has light emitting diodes of two or more types that emit light each having a different wavelength, and is placed along a side line of the titanium roller, the titanium roller and the light irradiation section are provided at a tip end side of the holding section, and the light irradiation section is placed at a rear end side rather than a rotating shaft of the titanium roller to irradiate in a tip end direction and irradiate the titanium roller with part of the irradiated light.

7. The beauty device according to claim 6, wherein the light emitting diodes include at least a light emitting diode that emits light having a wavelength ranging from red to yellow and a light emitting diode that emits near infrared light.

8. The beauty device according to claim 6, wherein each light emitting diode blinks at a frequency of two or more per second.

9. The beauty device according to claim 6, wherein the titanium roller is formed such that a surface of pure titanium is anodized.
